(19) United States
(12) Patent Application Publication
(10) Pub. No.: US 2025/0170105 A1
(43) Pub. Date: May 29, 2025

(54) ASSOCIATION WITH ANTIBIOTIC ACTIVITY, PHARMACEUTICAL COMPOSITIONS CONTAINING IT AND THEIR USE IN THE TREATMENT OF INFECTIONS

(71) Applicant: VETAGRO INTERNATIONAL S.R.L., Reggio Emilia (RE) (IT)

(72) Inventor: Ester GRILLI, Reggio Emilia (RE) (IT)

(21) Appl. No.: 18/846,620

(22) PCT Filed: Mar. 28, 2023
(86) PCT No.: PCT/IB2023/053079

§ 371 Date: Sep. 12, 2024

(30) Foreign Application Priority Data
Mar. 28, 2022 (IT) 102022000006047

(51) Int. Cl. A61K 31/43; A61K 31/05; A61K 31/165; A61K 31/19; A61K 31/192; A61K 31/194; A61K 31/195; A61K 31/35; A61K 31/496; A61K 31/505; A61K 31/546; A61K 31/635; A61K 31/65; A61K 31/7036; A61K 31/7048; A61K 38/12; A61P 33/02

(52) U.S. Cl. CPC A61K 31/43; A61K 31/05; A61K 31/165; A61K 31/19; A61K 31/192; A61K 31/194; A61K 31/195; A61K 31/35; A61K 31/496; A61K 31/505; A61K 31/546; A61K 31/635; A61K 31/65; A61K 31/7036; A61K 31/7048; A61K 38/12; A61P 33/02

(57) ABSTRACT

It is an object of the present invention a synergistic association with antibiotic activity, pharmaceutical compositions containing it, and their use in the prevention and treatment of infections in humans and animals.

It is an object of the present invention a synergistic association with antibiotic activity, pharmaceutical compositions containing it, and their use in the prevention and treatment of infections in humans and animals.

### TECHNICAL BACKGROUND

Resistance to an antibiotic, also called “antibiotic resistance”, is an adaptive phenomenon through which a microorganism manages to acquire the ability to survive an antibacterial agent that was initially able to inhibit growth or kill said microorganism.

The overuse of antibiotic drugs as well as their misuse, both in human and veterinary therapy and in animal husbandry, is one of the causes of the emergence and spread of resistant bacterial strains.

It is clear, therefore, that antibiotic resistance is recognized as a global public health priority because it carries important implications not only from a clinical but also from an economic perspective.

For this reason, the scientific community has long been searching for therapeutic alternatives to antibiotic therapy for the treatment of bacterial infections. Among the various alternatives, some organic acids (OAs) and/or natural or synthetic essential oils, and/or some molecules of plant origin, such as, for example, some molecules of plant origin reproduced by synthesis, also referred to as “natural identical compounds” (NICs), have shown effective antibacterial action and are therefore currently used in human and veterinary therapy but also, and especially, in the nutrition of livestock.

The same issues must also be addressed with respect to coccidiosis, which is an intestinal disease caused by protozoa that is particularly serious and present on poultry farms.

The paper Hongbin Si et al, (XP-002687332) concerns the antibacterial effect of oregano essential oil alone and in combination with antibiotics against broad-spectrum beta-lactamase-producing E. coli.

The paper Oliva Alessandra et al, (XP-055973526) concerns an extended investigation of tea tree oil (TTO) against a number of multidrug-resistant (MDR) microorganisms, particularly Gram-negative bacteria and methicillin-resistant Staphylococcus aureus.

WO 2021/114201 A1 refers to compositions containing at least one essential oil or at least one essential oil extract with broad-spectrum antibacterial activity.

The paper Giulia Giovagnoni et al, (XP-055973294) refers to a study to evaluate, in vivo, the antimicrobial potency of conventional antibiotics against B. hyodysenteriae and MCFAs (C6-C12 medium-chain fatty acids with antimicrobial activity) against a deposited strain isolated in northern Italy, including ATCC 27164 strain. The paper Bozkurt M. et al, (XP-055973593) concerns oregano essential oil (OEO) as a potential candidate for the control of coccidiosis in chickens. The objective of the study is to determine whether OEO and an approved anticoccidial, monensin sodium (MON), as supplements in feed can create synergy when combined at low dosages.

However, there is a constant need to find new alternatives to traditional antibiotic therapy, which would allow the dosage and/or frequency of administration of commonly used antibiotics to be limited and thus minimize their use, while at the same time exerting potent antibacterial action and preventing the development of antibiotic resistance.

### Aims of the Invention

It is an aim of the invention to provide an association of active ingredients having potent antibacterial and anticoccidial activity to reduce the use of conventional drugs and to limit the risk of the development of antibiotic resistance.

It is another aim of the invention to provide pharmaceutical compositions comprising the said association and their use in animal and human therapy.

### DESCRIPTION OF THE INVENTION

The Applicant, after extensive and intensive research and development activity, has surprisingly found that some specific molecules, including some natural identical compounds (NICs), are capable of significantly enhancing the activity of certain antibiotics and exerting a powerful and synergistic antibacterial action against specific genera of bacteria. In addition to natural identical compounds (NCIs), natural or synthetic essential oils, such as garlic oil, thyme oil, oregano oil, and rosemary oil, which contain, among others, thymol, carvacrol, eugenol etc., are also of interest.

Thus, according to one of its aspects, it is an object of the invention a synergistically acting pharmaceutical composition or association comprising or, alternatively, consisting of:
(i) at least one compound selected from the group comprising or, alternatively, consisting of:
   at least one organic acid, or a salt thereof, selected from the group comprising or, alternatively, consisting of a sorbic acid, citric acid, benzoic acid, hexanoic acid, lauric acid, and mixtures thereof;
   at least one terpene of plant origin, preferably selected from the group comprising or, alternatively, consisting of thymol, carvacrol, eugenol, and mixtures thereof; and
   monolaurin;
      in association with:
(ii) at least one antibiotic selected from the group comprising or, alternatively, consisting of colistin, amoxicillin, neomycin, tylosin, lincomycin, enrofloxacin, tetracycline, florfenicol, tiamulin, doxycycline and one of pharmaceutically acceptable salts thereof;
   for use in the treatment of bacterial infections caused by Brachyspira spp, E. coli spp, Salmonella spp, Campylobacter spp or Streptococcus spp, Vibrio spp.

Preferably, said compound (i) is selected from plant terpenes, even more preferably said compound (i) is selected from eugenol and thymol, and mixtures thereof; more preferably said compound (i) is thymol.

Preferably, said compound (i) is selected from natural or synthetic essential oils, such as thyme oil, oregano oil, rosemary oil, which also contain thymol, carvacrol, eugenol, among others.

Preferably, said compound (i) is selected from natural or synthetic essential oils, such as garlic oil.

Preferably, said antibiotic (ii) is selected from the group comprising or, alternatively, consisting of colistin, tetracycline, and neomycin, and mixtures thereof; more preferably said (ii) is colistin.

According to a preferred embodiment, said compound (i) is selected from plant terpenes, preferably from eugenol and thymol, and mixtures thereof, and said antibiotic (ii) is selected from the group comprising or, alternatively, consisting of colistin, tetracycline, and neomycin, and mixtures thereof.

According to an embodiment, it is an object of the invention synergistically acting pharmaceutical compositions or associations comprising or consisting of at least one or more of the following:
a. tetracycline or one of pharmaceutically acceptable salts thereof and citric acid and/or a salt thereof;
b. tetracycline or one of pharmaceutically acceptable salts thereof and sorbic acid and/or a salt thereof;
C. tetracycline or one of pharmaceutically acceptable salts thereof and benzoic acid and/or a salt thereof;
d. tetracycline or one of pharmaceutically acceptable salts thereof and thymol;
e. tetracycline or one of pharmaceutically acceptable salts thereof and eugenol;
f. neomycin or one of pharmaceutically acceptable salts thereof and sorbic acid and/or a salt thereof;
g. neomycin or one of pharmaceutically acceptable salts thereof and benzoic acid and/or a salt thereof;
h. neomycin or one of pharmaceutically acceptable salts thereof and thymol;
i. neomycin or one of pharmaceutically acceptable salts thereof and eugenol;
j. colistin or one of pharmaceutically acceptable salts thereof and sorbic acid and/or a salt thereof;
k. colistin or one of pharmaceutically acceptable salts thereof and benzoic acid and/or a salt thereof;
l. colistin or one of pharmaceutically acceptable salts thereof and thymol;
m. colistin or one of pharmaceutically acceptable salts thereof and eugenol;
n. colistin or one of pharmaceutically acceptable salts thereof and hexanoic acid and/or a salt thereof;
for their use in a method of treatment of bacterial infections caused by Brachyspira spp, E. coli spp, Salmonella spp, Campylobacter spp or Streptococcus spp.

According to an embodiment, it is an object of the invention a synergistically acting pharmaceutical composition or association selected by the group comprising or, alternatively, consisting of associations (a) to (n), as defined above, for its use in a method for the prevention and/or treatment of infections caused by Brachyspira spp, E. coli spp, Salmonella spp, Campylobacter spp or Streptococcus spp.

According to an embodiment, it is an object of the invention a synergistically acting pharmaceutical association comprising or, alternatively, consisting of thymol and colistin and/or at least one of pharmaceutically acceptable salts thereof. According to a further aspect of the present invention, said synergistically acting pharmaceutical association consists of thymol and colistin and/or at least one of pharmaceutically acceptable salts thereof. Compounds (i) and antibiotics (ii) are well known to the technique.

For example, thymol, 2-isopropyl-5-methylphenol, is a natural identical compound (NIC) known for its antimicrobial action and for exerting no serious side effects; thymol occurs naturally in plants of the genus Thymus.

Colistin, is a polymyxin antibiotic formed from a mixture of cyclic colistin A and B polypeptides. Although very effective, colistin is not a widely used antibiotic because it has been related to a potential risk of nephrotoxicity. It is therefore understood that the possibility of harnessing the potent antibiotic properties of colistin by administering it in low doses, allowed by the concomitant administration of thymol that enhances its effects, represents an important technical advance in the field of antibiotic therapy.

As mentioned, colistin and the other antibiotics may be present in the association of the invention as such or, when chemically possible, in the form of one of pharmaceutically acceptable salts thereof, e.g., as sodium salt, phosphate salt, sulfate salt, and the like. Even where not specifically stated, the term “antibiotic” here also includes, when chemically possible, pharmaceutically acceptable salts.

The present invention considers of interest not only natural identical compounds (NICs) but also natural or synthetic essential oils such as garlic oil, thyme oil, oregano oil, and rosemary oil that contain, among others, thymol, carvacrol, eugenol etc.

In a preferred embodiment, the pharmaceutical association for use according to the present invention is characterized by the fact that said compound (i) is selected from natural or synthetic essential oils, preferably is selected from garlic oil, thyme oil, oregano oil, rosemary oil which also contain thymol, carvacrol, eugenol, among others; preferably said essential oils being in combination with (ii) at least one antibiotic selected from the group comprising or, alternatively, consisting of colistin, amoxicillin, neomycin, tylosin, lincomycin, enrofloxacin, tetracycline, florfenicol, tiamulin, doxycycline and one of pharmaceutically acceptable salts thereof, for use in the treatment of bacterial infections caused by Brachyspira spp, E. coli spp, Salmonella spp, Campylobacter spp or Streptococcus spp, Vibrio spp.

Studies conducted by the Applicant on a variety of traditional antibiotics associated with identical natural compounds or other molecules, e.g., organic acids known for their antibiotic activity, have shown that the specific associations of the invention, for example, but not limited to, the association of thymol and colistin, exert potent antibiotic activity. In fact, in vitro treatment of bacterial strains with the mixture of the two active ingredients at concentrations much lower than their respective minimum inhibitory concentrations (MICs) equally afforded potent antibacterial activity. Of particular relevance is the fact that, as will be shown in the Experimental Section that follows, component (i) allows the effective dosage of antibiotic (ii) to be reduced by up to 50%, achieving an equal antibacterial effect. As a result, with the association of the invention, it is possible to administer to a subject to be treated a dose of antibiotic, e.g. colistin, half the MIC dose, thus reducing on the one hand the side effects for the treated subject and on the other hand the potential occurrence of antibiotic resistance.

According to another of its aspects, it is an object of the invention the use of one or more of the compounds (i), e.g., thymol, to enhance the antibiotic activity of one or more of the antibiotic compounds (ii), e.g., colistin.

According to another of its aspects, it is an object of the invention a pharmaceutical composition comprising the association of the invention in all the above-mentioned embodiments, together with one or more pharmaceutically acceptable excipients and vehicles, for use in the prevention and/or treatment of bacterial infections caused by Brachyspira spp, E. coli spp, Salmonella spp, Campylobacter spp or Streptococcus spp, Vibrio spp.

According to the invention, prevention and treatment are understood to be directed to humans or animals.

According to another of its aspects, it is an object of the invention a synergistically acting pharmaceutical association comprising
(iii) a terpene compound of plant origin, preferably selected from thymol, carvacrol and eugenol; more preferably thymol; and
(iv) an antibiotic selected from narasin, salinomycin, and monensin and/or at least one of pharmaceutically acceptable salts thereof;

for use in the treatment and/or prevention of infections caused by Eimeria coccidia.

Advantageously, the associations and pharmaceutical compositions of the invention comprise or, alternatively, consist of thymol and colistin.

Advantageously, the associations and pharmaceutical compositions of the invention comprise or, alternatively, consist of thymol and colistin for use in a method for treating bacterial infections caused by Brachyspira spp, E. coli spp, Salmonella spp, Campylobacter spp or Streptococcus spp, Vibrio spp.

For example, the associations, and the compositions of the invention, are particularly useful in the prevention and treatment of bacterial infections or coccidiosis in livestock, such as poultry farms and mammalian farms, such as sheep, cattle and pigs.

The compositions of the invention can be in dosage unit form or in multi-dose form, depending on the intended use. It is evident, in fact, that for administration to humans or a single animal, it is more useful to have dosage units, while for administration to numerous animals, as is the case in livestock farms, it is more advantageous to have multi-dose compositions, to be fractionated as needed and added, for example, to feed or water.

According to another of its aspects, it is an object of the invention the associations and/or compositions of the invention for use in the prevention and/or treatment of bacterial infections or coccidiosis, said use being characterized by the fact that antibiotic (ii) or (iv), when associated with compound (i) or (ii), is administered at a lower dosage than the “conventional” dosage or MIC dosage, expressed as the minimum inhibitory concentration. Antibiotic (ii) or (iv) is preferably administered at a dosage ranging from 50% to 90%, more preferably from 50% to 70%, advantageously around 50%, with respect to the MIC dosage.

“MIC dosage” here means the minimum dosage of antibiotic that inhibits the growth and replication of a bacterial strain on the basis of which threshold values are defined to describe a microorganism as susceptible or resistant to the action of the antibiotic itself (https://www.eucast.org/).

According to another of its aspects, it is an object of the invention the associations and/or compositions of the invention for use in the prevention and treatment of bacterial infections or coccidiosis, said use being characterized by the fact that compound (i) or (iii) and antibiotic (ii) or (iv) are both administered at lower dosages than their respective conventional dosages when such compounds are taken individually and not in combination with each other. According to a preferred embodiment of this aspect of the invention, antibiotic (ii) or (iv) is preferably administered at a dosage ranging from 50% to 90%, more preferably from 50% to 70%, advantageously around 50% relative to the MIC dosage, and compound (i) or (iii) is administered at a dosage ranging from 25% to 90%, more preferably from 25% to 70%, advantageously 25% to 50% with respect to the MIC dosage.

According to another of its aspects, it is an object of the invention a method for the prevention and treatment of bacterial infections that comprises administering, to a subject in need thereof, an effective amount, as indicated herein, of the associations or compositions of the invention.

Compositions of the invention comprising the association of the invention together with pharmaceutically acceptable excipients and/or vehicles are obtained according to known procedures.

As an example, oral or parenteral compositions can be prepared, oral compositions being preferred.

Among the oral compositions, liquid formulations, such as solutions, suspensions, emulsions, syrups, and the like; solid formulations, such as agglomerated powders, granules, pellets, capsules, tablets, and the like; and semisolid formulations, such as pastes, gels, and the like, can be prepared.

Excipients and vehicles that can be used in the preparation of the compositions of the invention can be selected from those conventionally used in the field of pharmaceutical technology.

By way of example, water, hydro-alcoholic solutions, glycerin, polyethylene glycol, propylene glycol, N,N-dimethylacetamide, etc., along with any stabilizing agents, thickeners, surfactants, buffers, and flavoring and/or sweetening substances, can be used as vehicles for solutions. The solubility of the components can be increased by the choice of an appropriate pH and/or the addition of cyclodextrins and hydrophilic polymers, such as polyvinylpyrrolidone, and the complexes thus formed can also be lyophilized and redissolved in an appropriate liquid.

Suspensions can be prepared using water and hydrophilic polymers, such as methyl cellulose, hydroxyethyl cellulose and hydroxypropyl cellulose, along with surfactants such as polysorbates.

Emulsions or microemulsions are two-phase systems and can be prepared by mixing two non-miscible phases such as oil-in-water, water-in-oil, or multiple emulsions (oil-in-water-in-oil or water-in-oil-in-water). Emulsions generally require the addition of appropriate surfactants, according to methods known to the person skilled in the art.

Solid compositions can be prepared from powders, granulates, solid dispersions or pellets/beads, the latter can be derived from microencapsulation processes.

The main solid forms are in the form of sachets, capsules or tablets.

Sachets are powder or granule preparations that can be or osoluble or can be administered after solubilization or dispersion in an appropriate liquid, usually water.

Tablets are generally obtained by compression of powders, granules, or beads, formulated with or without excipients, including diluents, binders, disintegrants, lubricants, or components capable of modifying absorption in the stomach or intestines. Tablets may be uncoated, coated, effervescent soluble, dispersible, orodispersible, modified-release, gastroresistant, or for use in the buccal cavity.

Capsules can be prepared with hard or soft shells, usually made of gelatin and possibly film-coated. They may contain solids, liquids, or pastes and are in form of soft, rigid, modified-release, gastro-resistant, or wafer-like capsules.

Other solid forms may be in the form of chewing gum.

There are also lipid-based formulations that, depending on their composition, size, chemical characteristics, and basic lipid system, can be classified as lipid solutions, lipid suspensions, emulsions, microemulsions, nanoemulsions, self-emulsifying systems and self-microemulsifying systems, solid lipid nanoparticles, solid lipid dispersions, niosomes, and liposomes. As such, all these formulations are well known to the person skilled in the art and may contain conventional excipients and vehicles such as surfactants, lipids, water, solvents, co-solvents, polysaccharides, etc.

In view of the hydrophobic nature of some compounds (i) and (iii), e.g., thymol, and its consequent poor solubility in water, nanosuspension-type systems with casein, using the “pH-driven” method, nanoemulsions and oil-in-water (O/W) microemulsions, solid lipid particles, liposomes, encapsulation in cyclodextrins or in natural polymers, can be used for the preparation of the compositions of the invention.

The invention will now be described with reference to the following examples, given for illustrative purposes only and in no way limiting.

### EXPERIMENTAL SECTION

### Example 1

Evaluation of the Efficacy of Associations of Antibiotic with Natural Identical Compounds/Organic Acids

The purpose of the study was to evaluate whether natural identical compounds (NICs) and organic acids (OAs), known for their antibiotic activity, were able to synergistically enhance the activity of known antibiotics (ABs) against certain microorganisms. Specifically, the following were conducted:
single tests to evaluate the effect of individual components of the association (AB, NIC, OA) against a reference microorganism and establishing effective/ineffective dosages;
combination test, combining ineffective doses of AB with low doses of NIC or OA (only partially effective alone) to assess whether the efficacy of AB could be increased by the combination with NIC or OA.

### Materials and Methods

Bacterial strains of Streptococcus suis and Escherichia coli K88 were used in this study.

### Bacterial Strains

Strains were stored at −80° C. in appropriate media with glycerol, thawed and cultured according to conventional practices. Strain characteristics and culture media are given in Table 1 below:

TABLE 1

| Bacterial Strain | Type | Culture media | Culture Conditions |
|---|---|---|---|
| Streptococcus suis 701 | Field isolated strain | Liquid: Mueller Hinton | 37° C., 24 hours, |
| Streptococcus suis 702 | (from pigs) | broth + Ca + Mg + lysed | anaerobiosis with |
| | | horse blood (5%) | AnaeroGen ® envelopes. |
| | | Solid: Tryptone Soy | (Oxoid) |
| | | Agar (TSA) with 5% | |
| | | sheep blood | |
| Escherichia Coli K88 95 | Field isolated strain | Liquid: heart-brain | 37° C., 24 hours, |
| Escherichia coli K88 97 | (from pigs) | infusion medium | anaerobiosis |
| | | Solid: Heart-brain agar | |

### Compounds to be Tested and Stock Solutions

The ABs shown in Tables 2 and 3 below, all obtained from Aplha Aesar, were tested. The OAs used are shown in Tables 2 and 3 below, all obtained from Merck. The stock solutions were prepared appropriately and in appropriate way and media for the different strains and could contain 3.5% ethanol to increase solubility.

### Antibacterial Test

The tests were conducted using the microdilution method in a 96-well plate to be microtitered with the medium and culture conditions appropriate for the strains. Bacterial strains (10⁵-10⁶ CFU/ml) were incubated with the compounds to be tested, while control strains, when necessary, were grown in media containing 3.5% ethanol to exclude an inhibitory effect of any ethanol present in the stock solutions. After incubation, MIC was defined as the lowest concentration resulting in zero absorbance at 630 nm, recorded with Varioskan® LUX Multimode Microplat Reader (Thermo Fisher Scientific). For each test, the percentage of inhibition (%) was calculated with respect to to the relative control.

### Phase 1—Individual Tests

ABs were tested in the range 64-0.13 ppm, OAs in the range 100-78 mM and NICs in the range 7.5-0.06 nM. Table 2 shows the screening on S. suis 701 and, S. suis 702

TABLE 2

| | | |
|---|---|---|
| MIC values | S. suis 701 | S. suis 702 |

| | | | | |
|---|---|---|---|---|
| Ampicillin | 0.25 | ppm | 0.25 | ppm |
| Penicillin | 0.13 | ppm | 0.13 | ppm |
| Ceftiofur | 0.5 | ppm | 0.5 | ppm |
| Enrofloxacin | 0.5 | ppm | 0.5 | ppm |
| Florfenicol | 2 | ppm | 2 | ppm |
| Tetracycline | 64 | ppm | 2 | ppm |
| Doxycycline | 16 | ppm | 0.5 | ppm |
| Neomycin | 8 | ppm | 16 | ppm |
| Tylosin | 2 | ppm | >64 | ppm |
| Tiamulin | 4 | ppm | 1 | ppm |
| Amoxicillin | 0.5 | ppm | 0.5 | ppm |
| Citric acid | 50 | mM | 25 | mM |
| Sorbic Acid | 100 | mM | 50 | mM |
| Benzoic Acid | 50 | mM | 25 | mM |
| Thymol | 1.87 | mM | 1.87 | mM |
| Eugenolo | 3.75 | mM | 3.75 | mM |

Table 3 shows the screening on E. coli 95 and E. coli 97

TABLE 3

| | | |
|---|---|---|
| MIC values | E. coli 95 | E. coli 97 |

| | | | | |
|---|---|---|---|---|
| Ceftiofur | 1 | ppm | 2 | ppm |
| Enrofloxacin | 2 | ppm | 2 | ppm |
| Florfenicol | 16 | ppm | >64 | ppm |
| Tetracycline | >64 | ppm | >64 | ppm |
| Neomycin | >64 | ppm | >64 | ppm |
| Amoxicillin | >64 | ppm | >64 | ppm |
| Colistin | 32 | ppm | 32 | ppm |
| Trimethoprim | >32 | ppm | >32 | ppm |
| Sulfamethoxazole | >608 | ppm | >608 | ppm |
| Citric Acid | >100 | mM | >100 | mM |
| Hexanoic acid | 50 | mM | 50 | mM |
| Sorbic Acid | 50 | mM | 50 | mM |
| Benzoic Acid | 50 | mM | 50 | mM |
| Thymol | 1.87 | mM | 1.87 | mM |
| Eugenolo | 3.75 | mM | 3.75 | mM |

### Phase 2—Combination Test

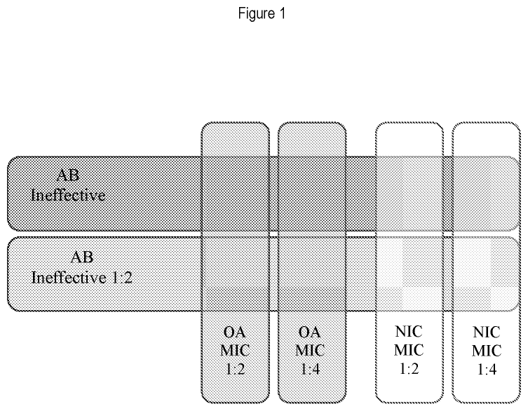

After phase 1, the strains were tested with associations at sub-effective doses of AB and OA or NIC in a 2×2 checkerboard model. Specifically, two sub-effective doses of AB were associated with two sub-effective doses of OA or NIC (half and one-quarter of MICs) as shown in FIG. 1.

### Results

The most representative results are reported below. The % indicate the percentage of inhibition (compounds alone and combinations).

### Abbreviations

TET tetracycline
NEO neomycin
COL colistin
CIT citric acid
SOR sorbic acid
BEN benzoic acid
THY thymol
EUG eugenol
HEX hexanoic acid

Tables 4 to 7 show the % of inhibition of the components of the associations alone, at the effective concentrations shown in Tables 2 and 3, and at the sub-effective concentrations (half and one-quarter of MICs), and the % of inhibition of the associations.

As will be seen, the specific associations provide significantly higher inhibitions than those of the individual components, and well above the additive effect.

TABLE 4
S. suis 701

| % inhibition | | CIT25 | CIT12.5 | SOR50 | SOR25 | BEN25 | BEN12.5 | THY0.94 | THY0.47 | EUG1.87 | EUG0.94 |
|---|---|---|---|---|---|---|---|---|---|---|---|
| | | 36 | 18 | 56 | 30 | 51 | 22 | 39 | 0 | 27 | 6 |
| TET32 | 28 | 95 | 73 | 88 | 67 | | 51 | 86 | 43 | | |
| TET16 | 11 | 98 | 28 | 74 | 50 | | 40 | 64 | 27 | | |
| NEO4 | 15 | | | 100 | 98 | 100 | 97 | 99 | 94 | 78 | 75 |
| NEO2 | 6 | | | 100 | 64 | 90 | 41 | 100.73 | 31 | 78 | 22 |

TABLE 5
S. suis 702

| % inhibition | | CIT12.5 | CIT6.25 | SOR25 | SOR12.5 | BEN12.5 | BEN6.25 | THY0.94 | THY0.47 | EUG1.87 | EUG0.94 |
|---|---|---|---|---|---|---|---|---|---|---|---|
| | | 43 | 12 | 38 | 26 | 39 | 21 | 36 | 10 | 12 | 9 |
| TET1 | 27 | 100 | 100 | 100 | 78 | 92 | | 78 | 41 | 72 | |
| TET0.5 | 20 | 85 | 42 | 81 | 53 | 53 | | 30 | 33 | 33 | |
| NEO8 | 16 | | | 100 | 86 | 90 | | 98 | 99 | 80 | 77 |
| NEO4 | 10 | | | 82 | 41 | 47 | | 99 | 24 | 62 | 20 |

TABLE 6
E. coli 95

| % inhibition | | THY 0.94 | THY 0.47 | SOR 25 | SOR 12.5 | BEN 25 | BEN 12.5 | EUG 1.87 | EUG 0.94 | HEX 25 | HEX 12.5 |
|---|---|---|---|---|---|---|---|---|---|---|---|
| | | 18 | 7 | 85 | 69 | 90 | 63 | 44 | 31 | 83 | 76 |
| COL 16 | 40 | 100 | 100 | 98 | 100 | 96 | 98 | 95 | 60 | 97 | 99 |
| COL 8 | 8 | 68 | 61 | 98 | 89 | 96 | 72 | 87 | 63 | 98 | 81 |
| COL 4 | 3 | 47 | 52 | 99 | 80 | 97 | 66 | 66 | 57 | 98 | 80 |

TABLE 7
E. coli 97

| % inhibition | | THY 0.94 | THY 0.47 | SOR 25 | SOR 12.5 | BEN 25 | BEN 12.5 | EUG 1.87 | EUG 0.94 | HEX 25 | HEX 12.5 |
|---|---|---|---|---|---|---|---|---|---|---|---|
| | | 12 | 8 | 93 | 79 | 97 | 79 | 34 | 26 | 83 | 65 |
| COL 16 | 29 | 100 | 100 | 100 | 100 | 100 | 100 | 100 | 100 | 100 | 100 |
| COL 8 | 12 | 40 | 32 | 100 | 98 | 100 | 100 | 100 | 95 | 100 | 99 |
| COL 4 | 10 | 24 | 24 | 100 | 85 | 100 | 95 | 100 | 52 | 100 | 95 |

### Example 2

Evaluation of the efficacy of antibiotic associations with identical natural compounds against coccidiosis. Anticoccidic tests were performed using a model that brings together a host cell line and Eimeria spp and allows the determination of the anticoccidic property of a given compound such as the ability to prevent Eimeria invasion within cells. More specifically, Madin-Darby Bovine Kidney (MDBK) cells and Eimeria tenella sporozoites were used as host cells and coccidia in this study. MDBK cells were seeded (1×10⁵ cells/well) on 24 wells and cultured to confluence for 48 hours in basal medium containing Dulbecco's Modified Eagle's Medium, 10% fetal bovine serum, 1× penicillin-streptomycin and L-glutamine 10 mM. Cells were incubated at 37° C. and with 5% CO₂. The coccidia inoculum was prepared according to the protocol described by Shirley in “Biotechnology Guidelines on techniques in coccidiosis research” with some modifications. Sporocysts obtained by mechanical rupture were resuspended in ecystion medium (trypsin 0.25%, bile salts 5 mg/mL, MgCl2 2 mg/mL) at a final concentration of 1×10⁶ sporocysts/mL. The sporocysts were excised for 90 min at 41° C. Then, the released sporocysts were filtered with a 5 μm Pluristrainer®, washed by centrifugation (500×g, 10 min) and counted. Infection was performed by covering MDBK cells with 1 mL of medium containing Eimeria spp sporozoites alone (control) or with the treatment of interest. Each well was infected with 5×10⁴ sporozoites and the cells were incubated for 24 h at 37° C. and 5% CO₂. The invasion efficiency was determined by the extracellular sporozoite count and estimated by the following formula:

$$100 - [ ( \frac{number of sporozoites in the supernatant after treatment}{number of starting sporozoites} ) \times 100 ]$$

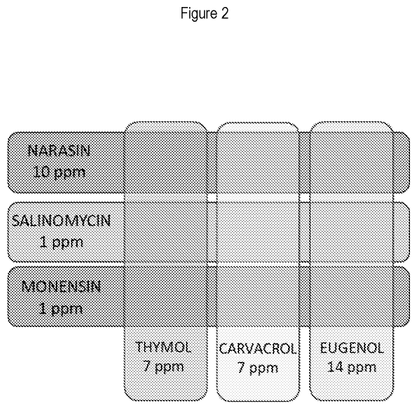

Associations of the antibiotics (iv) narasin (10 ppm), salinomycin (1 ppm), and monensin (1 ppm) with the compounds (iii) thymol (7 ppm), carvacrol (7 ppm), and eugenol (14 ppm), either individually or in combination, according to the scheme shown in FIG. 2, were tested.

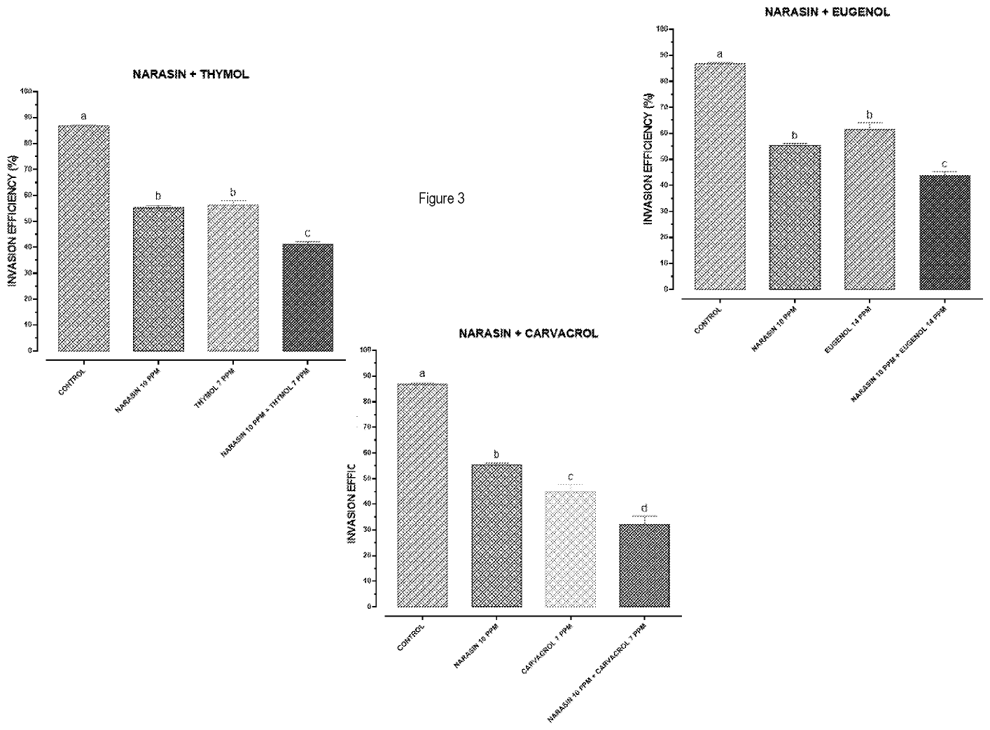

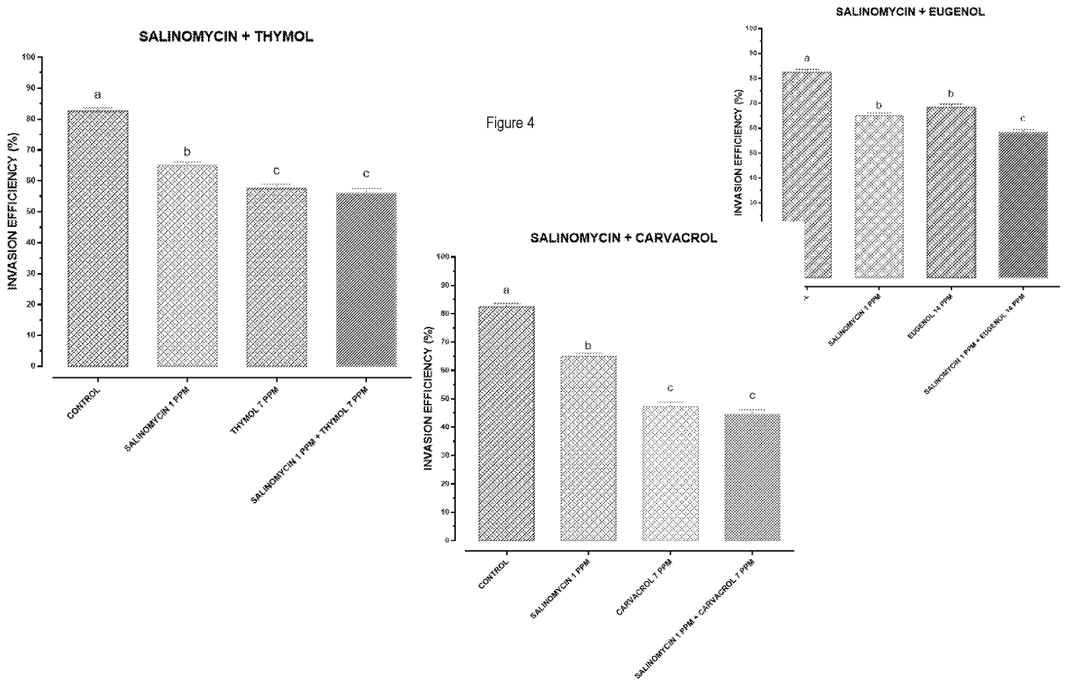

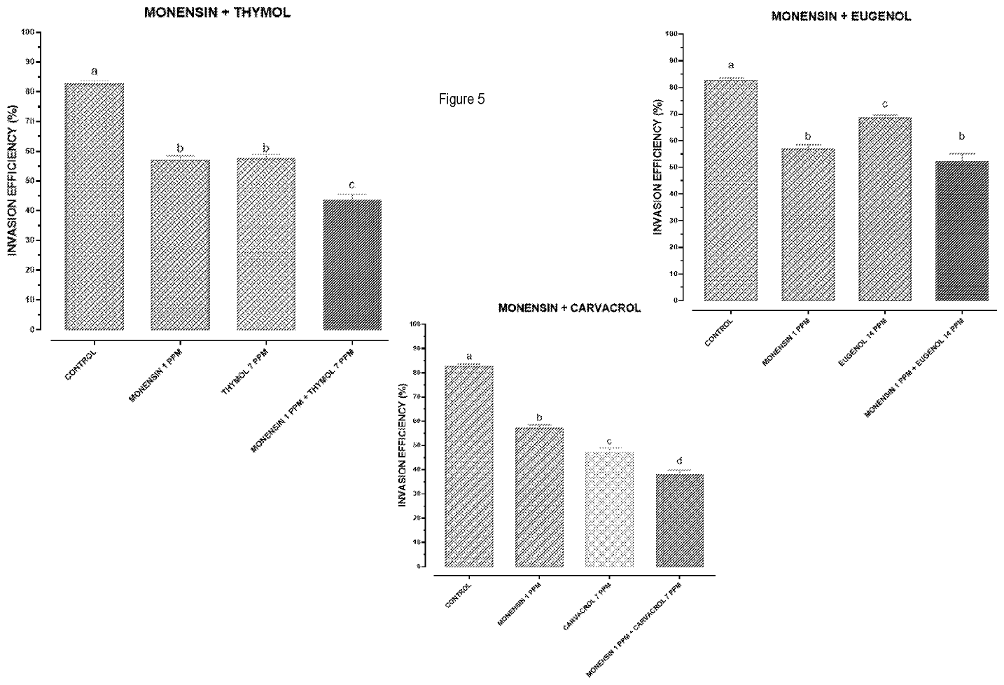

The results are shown in FIGS. 3 to 5. As can be seen from the said Figures, the associations showed significantly higher efficacy than efficacy relative to individual components (iii) and (iv).

1-13. (canceled)

14. A method of treatment comprising:
administering
(i) a compound selected from:
an organic acid selected from sorbic acid, citric acid, benzoic acid, hexanoic acid, lauric acid;
a terpene of plant origin selected from thymol, carvacrol, eugenol; and
monolaurin; and
(ii) an antibiotic selected from colistin, neomycin, tilosin, lincomycin, enrofloxacin, tetracycline, florfenicol, tiamulin, doxycycline and/or at least one of pharmaceutically acceptable salts thereof;
to a patient that has a bacterial infection caused by Brachyspira spp, E. coli spp, Salmonella spp, Campylobacter spp or Streptococcus spp, Vibrio spp.

15. The method according to claim 14, characterized by the fact that said compound (i) is selected from eugenol and thymol.

16. The method according to claim 14, characterized by the fact that said compound (i) is selected from natural or synthetic essential oils of garlic oil, thyme oil, oregano oil, rosemary oil that contain, among others, thymol, carvacrol, eugenol.

17. The method according to claim 14, characterized by the fact that said antibiotic (ii) is selected from colistin, tetracycline, and neomycin.

18. The method according to claim 14, characterized by the fact that it is selected from one or more of the following:
a. tetracycline or one of pharmaceutically acceptable salts thereof and citric acid;
b. tetracycline or one of pharmaceutically acceptable salts thereof and sorbic acid;
c. tetracycline or one of pharmaceutically acceptable salts thereof and benzoic acid;
d. tetracycline or one of pharmaceutically acceptable salts thereof and thymol;
e. tetracycline or one of pharmaceutically acceptable salts thereof and eugenol;
f. neomycin or one of pharmaceutically acceptable salts thereof and sorbic acid;
g. neomycin or one of pharmaceutically acceptable salts thereof and benzoic acid;
h. neomycin or one of pharmaceutically acceptable salts thereof and thymol;
i. neomycin or one of pharmaceutically acceptable salts thereof and eugenol;
j. colistin or one of pharmaceutically acceptable salts thereof and sorbic acid;
k. colistin or one of pharmaceutically acceptable salts thereof and benzoic acid;
l. colistin or one of pharmaceutically acceptable salts thereof and thymol;
m. colistin or one of pharmaceutically acceptable salts thereof and eugenol;
n. colistin or one of pharmaceutically acceptable salts thereof and hexanoic acid.

19. The method according to claim 18, characterized by the fact that it is selected from colistin or one of pharmaceutically acceptable salts thereof and thymol.

20. The method according to claim 14, wherein the compound and/or antibiotic are administered along with one or more pharmaceutically acceptable excipients and vehicles.

21. The method according to claim 14, wherein the patient that has a bacterial infection caused by E. coli spp or Streptococcus spp.

22. The method according to claim 14, characterized by the fact that the antibiotic (ii) is administered at a dosage lower than the MIC dosage.

23. The method according to claim 14, characterized by the fact that the antibiotic (ii) is administered at a dosage ranging from 25% to 90% with respect to the MIC dosage.

24. A method of treatment comprising:
administering
(i) a terpene of plant origin selected from thymol, carvacrol and eugenol; and
(ii) an antibiotic selected from narasin, salinomycin, and monensin and/or at least one of pharmaceutically acceptable salts thereof;
to a patient that has an infection caused by Eimeria spp.

25. The method of claim 24, wherein the terpene and antibiotic are administered along with one or more pharmaceutically acceptable excipients and vehicles.

26. The method of claim 24, wherein said composition is a formulation for oral administration.

27. A composition comprising:
(i) a compound selected from:
an organic acid selected from sorbic acid, citric acid, benzoic acid, hexanoic acid, lauric acid;
a terpene of plant origin selected from thymol, carvacrol, eugenol; and
monolaurin; and
(ii) an antibiotic selected from colistin, neomycin, tilosin, lincomycin, enrofloxacin, tetracycline, florfenicol, tiamulin, doxycycline and/or at least one of pharmaceutically acceptable salts thereof;
to a patient that has a bacterial infection caused by Brachyspira spp, E. coli spp, Salmonella spp, Campylobacter spp or Streptococcus spp, Vibrio spp.
